(19) United States
(12) Patent Application Publication
(10) Pub. No.: US 2019/0148000 A1
(43) Pub. Date: May 16, 2019

(54) METHOD AND SYSTEM FOR ASSESSING MOBILITY OR STABILITY OF A PERSON

(71) Applicant: Danny DRESSLER, Bromma (SE)

(72) Inventor: Danny DRESSLER, Bromma (SE)

(21) Appl. No.: 15/559,329

(22) PCT Filed: Mar. 18, 2016
(86) PCT No.: PCT/EP2016/056031

§ 371 Date: Sep. 18, 2017

(30) Foreign Application Priority Data
Mar. 18, 2015 (SE) 1550325-3

(51) Int. Cl. G16H 20/30; G16H 50/20; G06F 3/01; G06K 9/00; G06K 9/62; G06N 7/00; G06T 13/40

(52) U.S. Cl. CPC G16H 20/30; G16H 50/20; G06F 3/011; G06T 13/40; G06K 9/6263; G06N 7/005; G06K 9/00335

(57) ABSTRACT

A computer-assisted system and method for optimizing recommendations in a system for assessing mobility or stability of a subject comprising the steps of: a) using a video camera to create movement data, b) score movement data, c) based on the score of step b) and using a statistical model, create a recommendation and confidence data for the recommendation, d) provide the recommendation and the confidence data to a user, and e) modify the statistical model based on further user input.

### CROSS REFERENCE TO RELATED APPLICATION

The present invention is a nationalization application of International Application No. PCT/EP2016/082072, filed Mar. 18, 2016, corresponding to International Publication No. WO 2016/146834 A1, based upon an original priority Swedish Patent Application No. 1550325-3, filed Mar. 18, 2015, the subject matters of which are incorporated by reference herein.

### FIELD OF THE INVENTION

The present invention relates to method for providing and optimizing recommendations in a system for providing training advice or for assessing mobility or stability of a person.

### BACKGROUND OF THE INVENTION

There is a need for improved aids in providing training advice for athletes as well as non-athletes. Currently, training advice is provided by, for example, personal trainers. This is quite expensive and requires making an appointment, which is inconvenient.

Moreover, patients with impaired mobility or stability may improve if they carry out certain simple movements exercises.

The prior art describes various methods for automatically analyzing movements of patients with impaired mobility. For example, WO2009111886 provides a system for providing automatic treatment recommendations for patients with impaired mobility.

### SUMMARY OF THE INVENTION

In a first aspect of the invention, there is provided a computer-assisted method for optimizing recommendations in a system for assessing mobility or stability of a user comprising the steps of: a) using a motion capture device to create movement data for a user, b) score the movement data, c) based on the score of step b) and using a statistical model, create a treatment or exercise recommendation for the user, d) provide the treatment or exercise recommendation to the user, and e) modify the statistical model based on further user input. The treatment recommendation can be an exercise recommendation.

One advantage is that the method according to the invention provides feedback regarding the outcome of provided treatment recommendations, in order to improve future treatment recommendations.

In a preferred embodiment, the treatment recommendation is provided together with confidence data for the recommendation.

This has the advantage that the method provides information to the users about the confidence of the recommendations. The confidence changes according to user feedback. The confidence data provides information about how reliable the recommendation is. This is useful since the user will know how trustworthy the recommendation is.

The further user input step e) can be a second set of movement data regarding the same user, recorded at a later time point.

In one embodiment of the present invention, the movement data created in step a) and the treatment recommendation of step c) is provided to a second user and the further user input in step e) is a rating of the treatment recommendation of steps c) made by the second user. This provides fast learning of the system in the beginning. The movement data provided to the second user may then include a virtual avatar. The virtual avatar is preferably such that the first user cannot be identified. This has the advantage of protecting the privacy of the first user. The movement data is preferably provided to the second user in an anonymous form, so that the first user cannot be identified by the second user. Thus, the name and other personal details of the first user may not be available to the second user, and the movement data may include an avatar as described hereinabove. This also protects the privacy of the user.

The modification of the statistical model may be based on Bayes theorem.

The method may be used for optimizing recommendations in a system for assessing mobility or stability of a user or for optimizing recommendations in a system for providing training advice. The method may be such that is carried out only for non-medical purposes, i.e., non-therapeutic and non-diagnostic purposes, such as for providing fitness and training recommendations.

In a second aspect of the present invention, there is provided a system for carrying out the method according to the invention. Thus, there is provided a system for optimizing recommendations in a system for assessing the mobility or stability of a user or for providing training advice to a user comprising a motion capture device for creating movement data, a classifier for scoring the movement data and a statistical model for providing treatment recommendations selected from a library of treatment recommendations, where the system is adapted to create movement data of the user, to score the movement data of the user, and, based on the score, provide a treatment recommendation to the user together with confidence data for the recommendation, and where the statistical model can be modified based on further user input.

Briefly, the method according to the present invention is described as follows: a user is positioned in front of a motion capture device and performs certain movements. The motion capture device may comprise a video camera. The system analyses the movements and makes treatment recommendations based on a statistical model. The recommendation is provided to the user together with information about the statistical confidence for the recommendation. The statistical model is then modified by input provided by a user. This improves the statistical model for the recommendations so that it can make better predictions in the future. For example, new movement data regarding the same user recorded at a later time point can be used to provide feedback to the system. Alternatively, the recommendations and the movement data can be provide to second person who is an expert. The expert is allowed to access the movement data regarding the first person, together with recommendation. The expert is allowed to rate the recommendation, for example, to provide information about whether he or she agrees with the recommendation of the system. This input is used to modify the confidence of the recommendation of that particular type of assessment when the particular type of assessment is carried out for a new user of the system.

The system may provide a user account structure. The user account structure may provide for user names and passwords. Different types of accounts in the account structure may provide access to different types of information or may provide different authorizations.

The system may compromise an account structure with two different types of accounts, a first type of account that allows the creation of movement data and access to that movement data, but not rating recommendations, and a second type of account that allows access to movement data and rating of recommendations. The movement data may be provided to the second type of account in an anonymous form.

At a later stage, the fully taught system can be used by non-expert users to provide sophisticated feedback to a patient, i.e., a normal person can be “equipped” with the knowledge the system possesses.

### BRIEF DESCRIPTION OF THE DRAWINGS

FIG. 1 shows an overview of a system according to the invention;

FIG. 2-5 are flowcharts that schematically show the method according to the invention; and

FIG. 6 shows a display showing a recommendation.

### DETAILED DESCRIPTION OF THE INVENTION

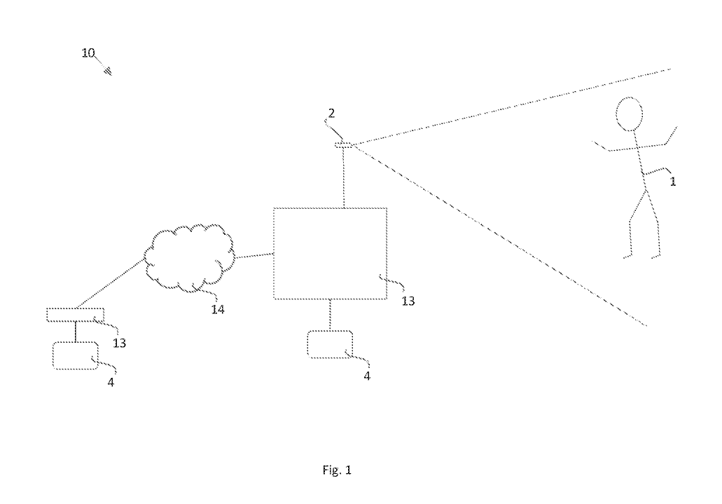

FIG. 1 shows how a user 1 uses the system 10 for carrying out the inventive method by performing movements in front of a motion capture device 2. The motion capture device is able to capture the movements of the user 1 from a distance from the user 1. The system 10 comprises at least one computer 13. The system, however, may comprise two or more computers 13 connected by a network 14, such as the Internet. At least one computer in the system has a user interface 3, which preferably comprises a display 4.

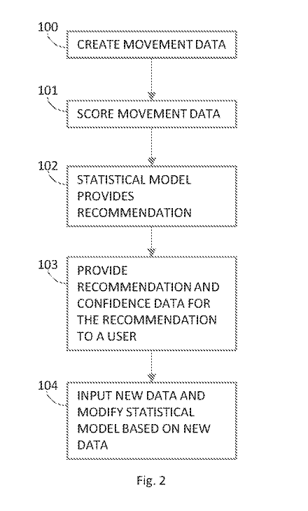

With reference to FIG. 2, in a first step 100 of the inventive method the system 10, which comprises a motion capture device 2, captures the movements of a user 1, and creates movement data. The user 1 is a person for whom mobility is to be assessed. The user 1 can, for example, be a patient with decreased mobility in the hamstrings, which can lead to pain and injury in the back. However, it should be noted that other persons, for example, a personal trainer, a physician or a physiotherapist, may be present and aid the user and provide instructions. Preferably, the movements that are carried out by the user 1 are predefined movements, such as, for example, toe touch, deep squat, etc. The system 10 may provide cues or instructions (text or synthetic voice) to the user 1 regarding which movements are to be carried out.

This step 100 can be carried out by methods that are known to a person skilled in the art. For example, the motion capture device 2 can be a video camera that records visible light or infrared (IR). The video camera may create a video, which is processed to create movement data. For example, pattern recognition software may be carried out to recognize the body of the user 1 and the movements carried out by him or her.

In a preferred embodiment, a time of flight camera is used as the motion capture device 2. The advantage of such a camera is that it provides 3D data. An example of a motion capture device 2 with a time of flight camera is the Microsoft Kinect camera. This type of device can capture motion and create movement data.

The creation of movement data may involve the generation of a virtual avatar that can be shown on a display 4. A virtual avatar is a moving visual and digital representation of the body of the user 1. The virtual avatar may be a skeleton, a stick figure or a ghost-like representation. The avatar may, for example, be visibly composed of sticks, spheres or cubes. Preferably the virtual avatar is such that the user cannot be identified. Preferably the face of the user cannot be identified on the avatar.

The output of this step 100 is referred to as movement data. The movement data may be stored in a MySQL database or a MONGO database.

The next step, step 101, is a scoring step, in which the movements performed by the user 1 are scored. A self-learning classifier 8—which may comprise a neural network computing device or lazy classifier, such as a k nearest neighbor algorithm implementing device—compares the movement data with a data set 7 for that particular movement (toe touch, deep squat, etc.). The classifier 8 may be based on the Weka suite of machine learning software.

The scoring in the system 10 will result in a score for each of the movements. The movements of the movement data may, for example, be scored as normal or non-normal. Alternatively, the movements may be scored as poor, average or optimal. The score may be stored as a numerical value, such as 1=poor, 2=average and 3=optimal.

Movement data will be added to the dataset 7 so that it increases. This will improve the classifier 8 over time as more and more data is added to the dataset.

In step 102, the system 10 creates at least one training or treatment recommendation for the user. Treatment may be issued only if the movement is scored as non-normal. Thus if the movement is healthy, no recommendation may be issued. Alternatively, if a movement is scored as normal, the system may instead provide recommendations to improve fitness. These might be certain exercises or activities, such as running or swimming. The user can also select goals (for example, fitness goals) that may be considered by the system to modify the recommendations.

The recommendation is based on the type of movement and the score of the movement, but may also take other parameters into account, such as, but not limited to, age, gender, BMI or previous or current illnesses. These parameters may be tied to a user account for the user and stored as user profile data 5. The user accounts may be provided in a user account structure that provides for user names and passwords. Preferably, each user account has a name and a password.

Treatment recommendations may include a regime for carrying out certain physical exercise, such as which exercises should be carried out and how often. The recommendation may include a time interval for carrying out the exercises, such as once per week. Other possible recommendations include physiotherapy or massage. The goal of the recommendation is to improve the fitness or the mobility or stability and/or reduce pain of the user.

The recommendations are generated by statistical model 9. The statistical model 9 may be a probabilistic network. The statistical model 9 selects a recommendation from a library 11 of possible recommendations. The statistical model 9 may select the recommendation in the library 11 with the highest probability of being correct, i.e. having a high probability of causing improvement for the user. The probability for the recommendation to be correct is determined by the statistical model 9. For this purpose, the system 10 may use conditional probabilities ((dynamic) Bayesian networks), as described below.

The system 10 may apply a cutoff for providing a recommendation such that a recommendation must have at least a minimum predetermined confidence in order for the recommendation to be provided to the user. The cut off may be from 60% to 90%, more preferably from 70% to 80%, and most preferably 75% probability of being correct. If no recommendation with appropriate confidence can be issued, the system may provide the recommendation with the highest available confidence. The user will then be informed that the provided recommendation is not verified and might not result in positive effects for the user.

In case the confidence is below the threshold the user can receive instructions to provide additional data to the system 10. The additional data can be used to improve the data set used in the analysis.

In step 103, the recommendation is provided to the user. This may be done via a user interface 3, for example, by displaying text and/or images on a display, synthetic voice, printing a report etc. The recommendation 103 may be provided to a physician or a physiotherapist that is present.

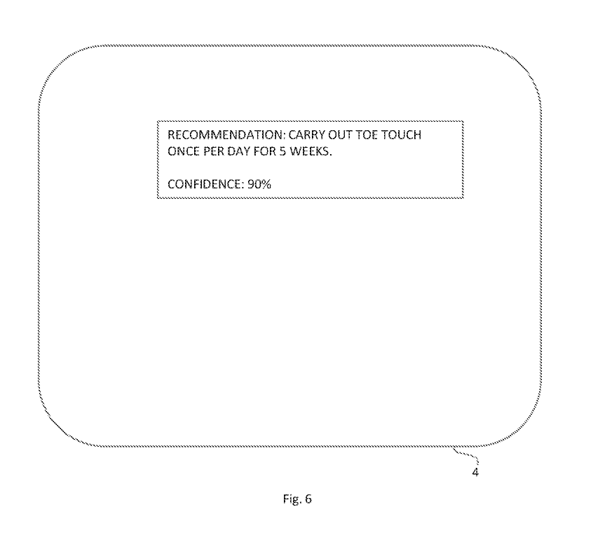

The recommendation may be provided together with confidence data for the recommendation. The confidence data provides information about how reliable the recommendation is. The confidence data is based on the probability for the recommendation to be correct as determined by the statistical model 9. Thus, the confidence data will reflect the probability that the recommendation will improve the conditions of the user. The confidence data may be provided as a percentage figure, as described for the cut-off probability, hereinabove. Alternatively, the confidence data may be displayed with a color scheme. For example, green may indicate a high confidence in the recommendation, yellow indicates intermediate confidence, and red indicates low confidence. For example, a green may represent a confidence of more than 85%, yellow may represent a confidence of from 75% to 85%, and red may represent a confidence of less than 75%. FIG. 6 schematically shows how a recommendation is provided on a display 4 of system together with a probability of how correct the recommendation is.

In step 104, the statistical model 9 is modified, by feedback learning, based on input of new data by a user, which can be the same user as in step 100, but can also be a different user. The feedback is arranged such that the statistical model will over time be improved and make recommendations that improve the mobility of users to a greater extent.

The statistical model 9 can be modified such that the probability that the same type of recommendation is issued a second time is increased. The statistical model 9 can be modified such that the probability that the same type of recommendation is issued a second time is decreased. “Same type of recommendation” refers to recommendations for the same type of movement with the same or similar score or similar user profiles (age, gender, BMI, previous illness, etc.) or combinations of these. The modification of the statistical model affects the confidence data provided to future users.

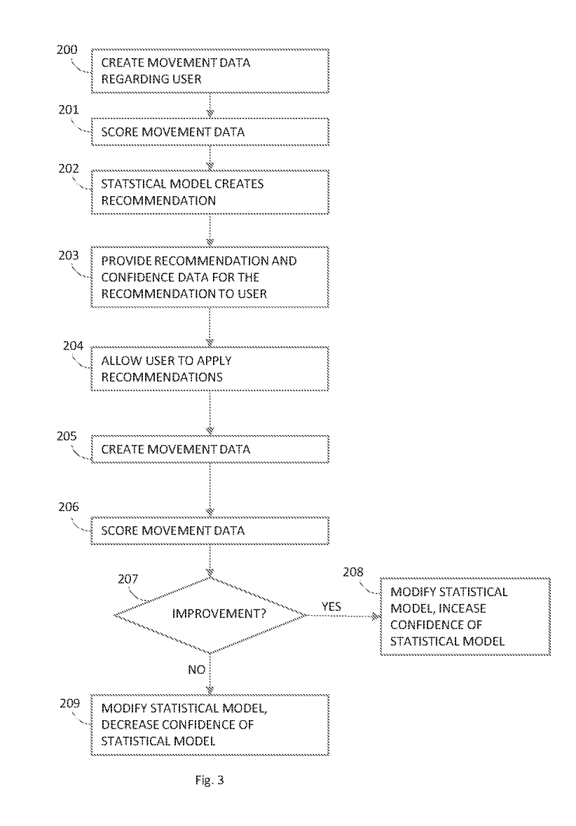

In a first preferred embodiment, step 104 is carried out as described in FIG. 3. Step 104 is then carried out as described in FIG. 3, steps 204 to 209. In FIG. 3, steps 200 to 203 are carried out as steps 100 to 103 in FIG. 1.

In the embodiment of FIG. 3, feedback learning is carried out as follows. In step 204, the user 1 is allowed to carry out the treatment recommendations made by the system 10 and provided to the user in step 203. Hopefully this results in an improvement of the condition of the mobility of the user (less pain, improved mobility). This may require some time to pass, to allow the user 1 to carry out the exercises. For example, a couple of weeks or months may be required. The time may be, for example, from 1 week to 10 weeks, depending on the movement, the movement score and the recommendation. However, the time may also be as short as a few minutes or hours, to allow the user to complete a few exercises. The system may be such that it automatically reminds the user 1 after this time has expired, for example, by email, or with a text message. This gives the user 1 a cue to make an appointment for making a second movement evaluation.

The user then, in step 205, again carries out movements and the movements are captured by motion capture device 2 to create a second set of movement data. This step is carried out as in step 200.

In step 206, the classifier 8 of the system 10 scores the movements. This is done in the same way as in step 201. Thus again, the movements may be scored as poor, average or optimal. The score created in step 206 is connected with the previous recommendation data created for the same user in step 202. Thus a logical association between the new score and the previous recommendation is created. This can be done with, for example, with an account structure with a login for each user.

This second score is used to provide feedback and to modify the statistical model 9 in steps 207 to 209. In step 207 the score of the movements of the user in step 201 is compared to the score of the movement of the user in step 206. If the condition of the user is better in step 206 that in step 201, i.e., the condition of the user has improved, the statistical model used in step 202 is modified in step 208 such that the probability for the system 10 to again issue the same type of recommendation increases. This may be done by simply comparing the numerical scores. If the score is higher the second time, an improvement has taken place. If the condition of the patient in step 206 is worse (i.e., the score is lower) than in step 201, the statistical model 9 is modified in step 209 such that the probability for the system 10 to again issue the same type of recommendation decreases.

Thereby, the statistical model 9 will be modified (preferably it has improved to be more accurate) the next time it is used (which may be by a completely different user).

However, an improvement may be relative. For a user with a certain illness where deterioration of the condition is expected, an unchanged condition or even a slower than expected worsening may in fact be an improvement (this is a relative improvement). Here the movement score of the user must be compared with a baseline that indicates the expected development of the user. A user 1 may in this case enter a certain medical condition or injury history, which may be considered by the system. Depending on the condition or injury history the system will choose, whether or not an unchanged condition can indicate a positive result.

The modification of the statistical model 9 may preferably be based on Bayes Theorem:

p(H|E)=p(E|H)×p(H)/p(E)

Bayes Theorem is used to evaluate how strongly a bit of evidence supports a hypothesis. This mathematical model provides a mechanism for the systems to update its confidence based on given evidence.

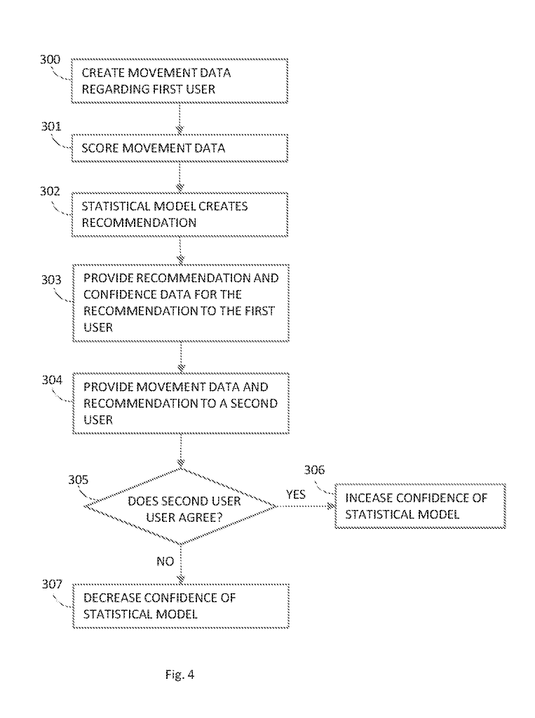

A second preferred embodiment is shown in FIG. 4. Here, step 104 is carried out as follows. As indicated above, an expert, for example, a physician or physiotherapist (which may be referred to herein as an expert user or a second user), may rate previously made recommendations made by system 10 as indicated in FIG. 4. Steps 300 to 303 are carried out as steps 100 to 103. However, step 303 is optional and may be omitted. The experts step 304 are provided, through user interface 3, with at least the movement data of step 300 (such as the virtual avatar) and the recommendation created in step 302 made for at least one user by the system 10.

The use of an avatar has the advantage that it preserves the privacy of the first user. This may be important, for example, for persons who have movement problems, or persons who carry out exercises in pain (see above). The expert user may optionally also be provided with other type of data specific for the first user, such as age, gender and BMI. The expert user may optionally also see the score of the movements made by the system 10 in step 301 (for example, poor, average or optimal).

The expert user will then provide input to the system 10 by using user interface 3, such as for example rate the previous recommendation. This input is used to modify the statistical model 9. For example, the second user may in 305 be able to state whether he or she agrees with the recommendation made by the system 10 in step 302. Rating may be done in any way. For example, a simple agree/does not agree scale can be used. Alternatively, a percentage or points may be used. This statement provides feedback that modifies the statistical model 9. The modification of the statistical model 9 may preferably be based on Bayes Theorem as described hereinabove.

If the expert user agrees with the recommendation made by the system 10, the probability that the same type of recommendation is issued a second time is increased in step 306. If the expert user does not agree with the recommendation issued by the system 10, the statistical model is modified such that the probability of the same type of recommendation is issued a second time is decreased in step 307.

Thereby, the statistical model 9 will be modified (preferably it has improved to be more accurate) the next time it is used (which may be by a completely different user). The system 10 may provide a type of user account (second or expert user-type account) that allows the rating of recommendations made by the system. By logging into the system using such an account, a second (expert) user is able to rate the recommendations. That type of account normally does not enable the creation of movement data. Thus, there may be two types of accounts: 1) first user-type accounts, that allow the creation of movement data but not rating of recommendations made by the system 10, and 2) second user-type accounts that allow the rating of recommendations made by the system 10.

Movement data is preferably presented to the second user type accounts in anonymous form. Movement data may be presented in anonymous form to second user-type accounts and in non-anonymous form to first user-type accounts.

When logging into the second user type-account, the expert user is presented with movement data and related recommendations previously made by the system, and the system 10 then allows the expert user to rate the recommendations. The movement data and associated recommendations may be selected for rating by the system 10. In particular, movement data and recommendations may be selected that are in need of verification (from a statistical point of view) by the system 10. Thus, treatment recommendations with a low confidence may be selected for rating by the system 10. Treatment recommendations below a threshold (see above) may be selected for rating.

The second type of account may provide movement data for a plurality of users.

The use of an expert user is particularly useful in the beginning of the use of the system, when the dataset and the number of users may be too small to create feedback learning at the desired rate.

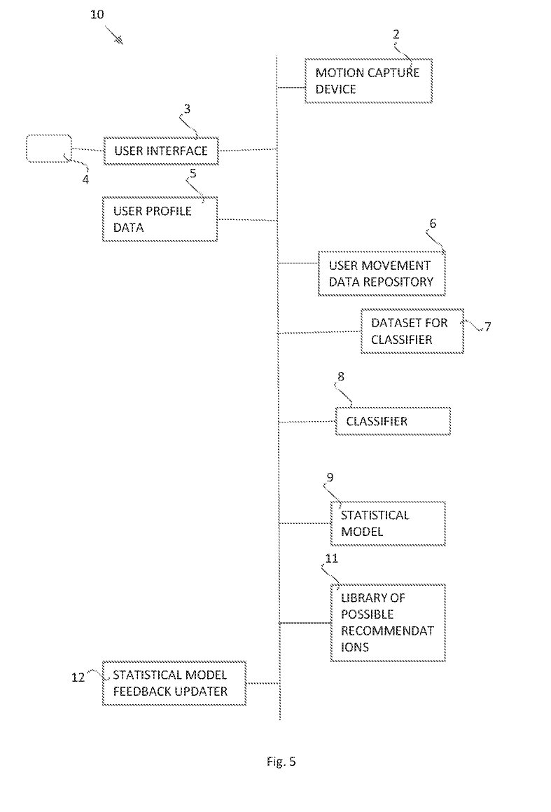

FIG. 5 is a schematic overview of some details of a system 10 that is able to carry out the method according to the invention. System 10 comprises a motion capture device 2 which has a functionality for creating movement data. Moreover, the system 10 may comprise a user interface 3 that may include a keyboard and a display 4. The system 10 has a memory that supports the various parts of the system 10.

The memory may store user profile data 5, such as name, log in data, age, gender, BMI, previous illness, etc. User profile data may also store recommendations issued by the system 10. The system also has a repository 6 for storing user movement data for at least one user 1. The system further comprises a dataset 7 for comparing with user movement data 6. The movement data 6 may be added to the dataset 7. This makes the dataset 7 grow over time, so that the confidence of the predictions increases over time. The comparison of movement data 6 with dataset 7 is carried out by classifier 8, which may be a neural network. The statistical model 9 is responsible for making treatment recommendations. The statistical model 9 chooses recommendations from a library of possible recommendations 11.

The statistical model 9 provides a probability for the success for at least one recommendation in the library 11. This probability states how probable it is that a certain recommendation will be successful for a certain condition (movement type, movement score, age, gender, BMI, previous illness, etc. or combinations thereof). This probability is feedback modified in step 104 by statistical model feedback updater 12.

The system 10 may be implemented using any suitable computer environment. Various parts of the system may distributed across different computers 13 connected by a network 14, such as the Internet. Communications within the system may be wireless or wire bound. The system may have more than one user interface 3 and more than one motion capture device 2.

While the invention has been described with reference to specific exemplary embodiments, the description is in general only intended to illustrate the inventive concept and should not be taken as limiting the scope of the invention. The invention is generally defined by the claims.

1-8. (canceled)

9. A computer-assisted method for optimizing recommendations in a system for assessing mobility or stability of a user comprising the steps of:
a) using a motion capture device to create movement data for a user;
b) score the movement data;
c) based on the score of step b) and using a statistical model, create a treatment recommendation for the user;
d) provide the treatment recommendation to the user together with confidence data for the recommendation; and
e) modify the statistical model based on further user input.

10. The method according to claim 9, wherein the further user input in step e) is a second set of movement data regarding the same user, and recorded at a later time point.

11. The method according to claim 9, wherein the movement data created in step a) and the treatment recommendation of step c) are provided to a second user, and the further user input in step e) is a rating of the treatment recommendation of step c) made by a second user.

12. The method according to claim 11, wherein the movement data provided to the second user includes a virtual avatar.

13. The method according to claim 9, wherein the modification of the statistical model is based on Bayes Theorem.

14. A system for carrying out the method according to claim 9.

15. The system according to claim 14, further comprising means to carry out the method according to claim 10.

16. The system according to claim 15, further comprising means to carry out the method according to claim 11.

17. The system according to claim 16, further comprising means to carry out the method according to claim 12.

18. The system according to claim 7, further comprising means to carry out the method according to claim 13.
